(19) United States
(12) Patent Application Publication
(10) Pub. No.: US 2002/0001613 A1
(43) Pub. Date: Jan. 3, 2002

(54) Method of manufacturing liposomes

(76) Inventors: Susan Niemiec, Yardley, PA (US); Glenn A. Nystrand, Lebanon, NJ (US); Jonas C.T. Wang, Robbinsville, NJ (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/779,069

(22) Filed: Feb. 9, 2001

Related U.S. Application Data
(60) Provisional application No. 60/181,019, filed on Feb. 8, 2000.

(51) Int. Cl. A61K 9/127; B01J 13/02

(52) U.S. Cl. 424/450; 264/4.1

(57) ABSTRACT

The present invention relates to a method of making a liposome, said method comprising the steps of: (a) mixing a lipophilic phase and a hydrophilic phase, said lipophilic phase comprising an amphiphilic bilayer-forming substance; and (b) applying a cavitational sheer force to said mixture to form said liposome; wherein said cavitational force is created by contacting said mixture with a member that vibrates at an ultrasonic frequency.

### FIELD OF THE INVENTION

The present invention relates to a method of producing liposomes using a process that employs ultrasonic cavitational forces to form the liposomes.

### BACKGROUND OF THE INVENTION

Liposomes were first described in 1965 by Bangham (Bangham, A. D., Standish, M. M. and Watkins, J. C. 1965. “Diffusion of Univalent Ions across the lamellae of swollen phospholipid,” J. Mol. Biol., 13: 238-252). Liposomes are classified by size, number of bilayers and hydrophobicity of the center core. A conventional liposome is composed of lipid bilayers surrounding a hydrophilic core. The lipids of the lipid bilayers can have conjugating groups such as proteins, antibody polymers, and cationic polyelectrolytes on the surface of the liposomes and will act as targeting surface agents. Lipid vesicles are often classified into three groups by size and structure; multilamellar vesicles (MLVs), large unilamellar vesicles (LUVs), small unilamellar vesicles (SUVs), and paucilamellar (PLVs) vesicles. MLVs are onion-like structures having a series of substantially spherical shells formed of lipid bilayers interspersed with aqueous layers. LUVs have a diameter greater than 1 μm and are formed of a single lipid bilayer surrounding a large hydrophilic core phase. SUVs are similar in structure to LUVs except their diameter is less than an LUV, e.g., less than 100 nm. PLVs are vesicles that have an internal hydrophobic core surrounded by bilayers. See, e.g., Callow and McGrath, Cryobiology, 1985 22(3) pp. 251-267.

Liposomes were initially used as models for studying biological membranes. However, in the last 15 years liposomal delivery systems have been designed as advanced delivery vehicles of drugs and other benefits agents into biological tissues. See, e.g., Gregoriadis, G., ed. 1988. Liposomes as Drug Carriers, New York: John Wiley, pp. 3-18). Liposomes have also been incorporated in a large variety of consumer products ranging from cosmetics to foods.

Traditionally, the thin-film method was used to manufacture liposomes. In this method, the bilayer-forming elements are mixed with a volatile organic solvent (such as chloroform, ether, ethanol, or a combination of these) in a mixing vessel (such as a round bottom flask). The predominant bilayer-forming element used to form conventional phospholipid vesicles is usually a neutral phospholipid such as phosphatidylcholine. Cholesterol is also often included to provide greater stability of the liposome in biological fluids. A charged species such as phosphatidylserine may also be added to prevent aggregation, and other elements such as natural acidic lipids and antioxidants, may also be included. The lipid-solvent solution is then placed under specified surrounding conditions (e.g., pressure and temperature) such that the volatile solvent is removed by evaporation (e.g., using a rotary evaporator) resulting in the formation of a dry lipid film. This film is then hydrated with aqueous medium containing dissolved solutes, including buffers, salts, and hydrophilic compounds, that are to be entrapped in the lipid vesicles. The hydration steps used influence the type of liposomes formed (e.g., the number of bilayers formed, vesicle size, and entrapment volume). If desirable, non-encapsulated drug or active can be removed from the mixture by a variety of techniques such as centrifugation, dialysis or diafiltration and recovered.

This film hydration method, however, is time consuming, involves the use of organic solvents, and scale-up is quite cumbersome. As a result, other processes for the preparation of liposomes have been used, including: (1) the injection of amphiphilic bilayer-forming substances, dissolved in organic solvents, into an aqueous medium (optionally containing a pharmaceutical substances) as described by Batrzri and Szoka (Batzri, S. and Korn, E. D., 1973, “Single Bilayer Liposomes Prepared without Sonication,” Biochim. Biophys. Acta, 298:1015-1019, Szoka, F. C. and Papahadjopoulos, D., 1980, Comparative Properties and Methods of Preparation of Lipid Vesicles (Liposomes), “Ann. Rev. Biophy. Bioeng., 9: 467-508); (2) the dissolution of amphiphilic, bilayer-forming substances in an aqueous medium using solubilization agents resulting in the formation of mixed micelles or associates, followed by the subsequent removal of the solubilization agent from the aqueous medium by means of gel chromatography or equilibrium dialysis (See, e.g., Milsmann, M. H. W., Schwender, R. A. and Weber, H., 1978, “The preparation of large scale bilayer liposomes by a fast and controlled dialysis,” Biochim. Biophys. Acta, 512: 47-155; and U.S. Pat. No. 4,687,661); and (3) the dispersing of amphiphilic bilayer-forming substances in water to form optically clear suspensions using high-pressure homogenization as described in Huang (See Huang, C. H., 1969, “Studies of phosphatidylcholine vesicles. Formation and Physical characteristics,” Biochemistry, 8:344-351).

These known processes, however, are also often unsuitable for large-scale preparation of liposomes as further separation processes, such as ultracentrifugation and/or fractional filtration, often must subsequently be carried out to achieve increased homogeneity, resulting in extended processing time. Furthermore, in regards to pharmaceuticals, optimum liposome preparations would avoid the use of organic solvents and detergents which are difficult to remove, exhibit high trapping efficiency, yield well-defined and reproducible liposomes, and be rapid and amenable to scale-up procedures.

In response to these needs, still other methods of preparing liposomes have been developed for large-scale manufacturing of liposomes (e.g., to be used as cosmetic or pharmaceutical products). For example, European Patent No. 753 340 A2 discloses a method of manufacturing liposomes using phospholipids in a high-speed rotary dispersing machine and U.S. Pat. No. 4,895,452 discloses a method that uses a shear mixing in a substantially cylindrical mixing chamber having at least one tangential input for rapid production of lipid vesicles.

Accordingly, the object of the present invention is to provide a method for making liposomes (e.g., that contain a benefit agent such as a drug), which lends itself to commercial, high-volume production.

### SUMMARY OF THE INVENTION

In one aspect, the invention features a method of making a liposome, the method comprising the steps of: (a) mixing a lipophilic phase and a hydrophilic phase, the lipophilic phase comprising an amphiphilic bilayer-forming substance and (b) applying a cavitational sheer force to the mixture to form the liposome. In one embodiment, the cavitational force is created by contacting the mixture (e.g., under pressure) with a member (e.g., a flat metal sheet such as a blade) that vibrates (e.g., the member vibrates via mechanical assistance or vibrates as a result of the mixture passing over it) at an ultrasonic frequency. In one embodiment, the lipophilic phase and the hydrophilic phases are mixed under pressure and/or passed through an orifice (e.g., having a diameter between about 0.0005 cm² to about 0.01 cm²). In one embodiment, the method further comprises the step of applying a second sheer force, wherein the second sheer force is created by passing the mixture through an orifice (e.g., a tuning valve).

Other features and advantages of the present invention will be apparent from the detailed description of the invention and from the claims

### BRIEF DESCRIPTION OF THE FIGURES

FIG. 1 is a schematic diagram of a manufacturing apparatus that can be used in an embodiment of the invention.

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

The present invention relates to a method of producing liposomes. The method is based on the shear mixing of a hydrophobic liquid phase and a hydrophilic liquid phase utilizing cavitational forces to rapidly hydrate the hydrophobic phase with the hydrophilic phase, thereby forming liposomal structures. What is meant by a liposomes is a vescicle having at least one lipid bilayer surrounding an inner liquid phase (e.g., either a lipid bilayer surrounding either a liquid core or a liquid phase dispersed between it and another lipid bilayers). The liposome may have various structures such as multilamellar (MLVs), unilamellar (LUVs or SUVs), and paucilamellar (PLVs) as discussed above. The resulting structure of the liposome is dependent, in part, on the choice of materials forming the hydrophobic phase and the manufacturing parameters such as pressure, temperature, and flow rates.

The method of the invention uses a means of bringing together two fluids, one hydrophilic in nature and one hydrophilic in nature, which are combined and exposed to a cavitational force. In one embodiment, the combined fluids are mixed and then forced under increased pressure (e.g., 2-10,000 psi) around a flat sheet, such as a thin edged blade, that vibrates in the ultrasonic range (e.g., from about 10,000 to 50,000 Hz). It is believed that as the fluid tries to fill the minute void space created on either side of the blade as it oscillates, zones of intense cavitation are generated, and the extremely high level of shear resulting from the violent collapse of thousands of tiny cavitation bubbles creates the lipid bilayers.

A benefit of this process is that the resulting liposomes may have small mean particle size (e.g., between 50 nm and 10 microns such as between 50 and 500 nm). Liposomes with larger mean particle size can experience separation during aging. Another benefit of this process is that small bubbles created during the preceding mixing step can be eliminated or suppressed from the mixture because of ultrasonic shear forces. Furthermore, as the agitation chamber of the apparatus may substantially only contain the two liquid phases materials, the method can substantially prevent entry of air (e.g., in the form of bubbles) into the resulting liposome mixture. The elimination of air from the resulting mixture protects oxygen sensitive materials (e.g., benefit agents such as retinol) from oxidation.

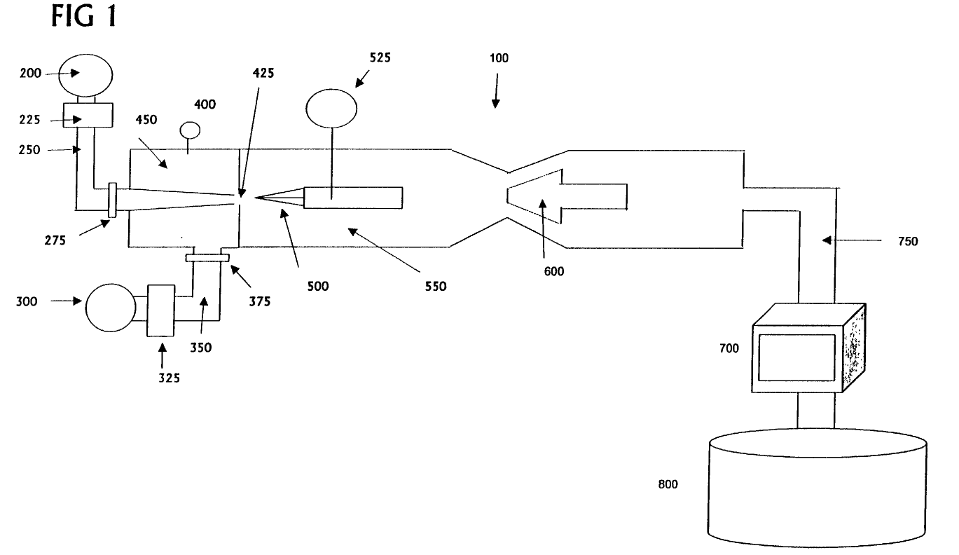

In one embodiment, the method utilize the Sonolator™ device Model No. A-HP made by Sonic Corporation, Stratford, Conn. as shown in FIG. 1 as apparatus 100. The water phase is stored in water reservoir 200 while the lipid phase is stored in lipid reservoir 300. The water phase and the lipid phase are pumped into a premixing chamber 450 respectively through water feed line 250 and lipid feed line 350 under pressure from their respective water positive displacement pump 225 and lipid positive displacement pump 325 (Triplex Piston Pumps Model No. 521, Cat Pumps Corp., Minneapolis, Minn.). Examples of other positive displacement pumps include, but are not limited to, plunger, gear, and centrifugal pumps. Flow rates can range from 0.25 to 600 gallons per minute. Pre-mixing chamber 450 has a pressure gauge 400 to measure the pressure with pre-mixing chamber 450. The pressure within pre-mixing chamber 450 may range between 2-10,000 psi. There is also a back pressure safety check valve 275 in the water feed line 250 and a back pressure safety check valve 375 in the oil feed line 350 to prevent back flow of the phases being pumped.

Once pumped into the pre-mixture chamber 450, the two fluids meet just before the orifice 425 leading to the mixing chamber 550. As the fluids travel through the orifice 425, the mixture experienced extreme cavitational shear forces resulting from the ultrasonic oscillations 10,000-50,000 Hz of the vibrating blade 500 inside the mixing chamber 550. The ultrasonic oscillations, created by the fluids passing over the blde, are measured via an acoustic intensity meter 525. The mixture experiences additional shear forces as it passes through the orifice of the tuning valve 600 at end of the mixing chamber before exiting mixing chamber 500 through exit tube 750. The tuning valve is adjusted to add a slight amount of backpressure (e.g., 1-2 psi) such that any Corrolis effect (twisting) of the flow stream impinging upon the blade 500 is straightened.

The mixture then passes through exit tube 750 into a heat exchanger 700 (Plate/Heat heat exchanger made by Vicarb. Inc., New market, Ontario, Canada) or series of heat exchangers (not shown) so that the desired temperature decrease of the mixture is obtained. Examples of heat exchangers include, but are not limited to, plate/frame, shell/tube, and/or sweep/scrape heat exchangers. The mixture is then collected in product reservoir 800.

Liposomes manufactured according to the present invention comprise at least one amphiphilic bilayer-forming substance and may comprise a benefit agent. The benefit agent may be contained either within the lipid bilayer or the hydrophilic or hydrophilic compartments of the liposome.

What is meant by amphiphilic bilayer-forming substance is a lipid that is comprised of both a hydrophilic and lipophilic group and is capable of forming, either alone or in combination with other lipids, the bilayer of a liposome. The lipid can have single or multiple lipophilic side chains being either saturated or unsaturated in nature and branched or linear in structure. The amphiphilic bilayer forming agent can be phospholipid or a ceramide.

Multiple lipophilic side chain amphiphilic bilayer-forming substances are amphiphilic bilayer-forming substances have two or more lipophilic side chains (e.g., that are attached to a polar head group). Such lipids may be nonionic, cationic, anionic, zwitterionic in nature. Examples of suitable multiple lipophilic side chain amphiphilic bilayer-forming substances include, but are not limited to, those bilayer-forming cationic lipids that contain two saturated or unsaturated fatty acid chains (e.g., side chains having from about 10 to about 30 carbon atoms) such as di (soyoylethyl) hydroxyethylmonium methosulfate (DSHM), N-[I-(2,3-dioleyloxy)propyl]-N,N,N-trimethylammonium bromide (DOTMA), 1,2-dimyristyloxypropyl-N,N-dimethyl-hydroxyethyl ammonium bromide (DMRIE), [N-(N,N′-dimethylaminoethane) carbamoyl] cholesterol (DC-Chol), dioctadecylamidoglycyl spermidine (DOGS), dimethyl dioctadecylammonium bromide (DDAB), dioleoyl phosphatidylethanolamine (DOPE), 2,3-dioleoyloxyl-N[2(sperminecarbozamide-O-ethyl]-N,N-dimethyl-propanaminium trifluoroacetate (DOSPA), I-[2-(oleoyloxy)-ethyl]-2-oleyl-3-(2hydroxyethyl) imidazolinium chloride (DOTIM), 1,2-dioleoyloxy-3-(trimethylammonio) propane (DOTAP), 1,2-diacyl-3-trimethylammonium propane (TAP), 1,2-diacyl-3-dimethylammonium propane (DAP), fatty acid salts of quaternary amines such as dicocodimonium chloride (Quaternium 34), and quaternary dimethyldiacyl amines wherein the acyl groups have from about 8 carbon atoms to about 30 carbon atoms (e.g., from about 10 carbon atoms to about 30 carbon atoms), and derivatives thereof such as ammonium derivatives, i.e. dimethyl dihydrogenated tallow ammonium chloride (Quaternium 18), and decyl dimethyl octyl ammonium chloride (Quaternium 24), and derivatives thereof. Other suitable cationic dual chain lipids are further described in the following references: Fasbender et al., 269 Am J Physiol L45-L5 1 (1995); Solodin et al., 34 Biochemistry 13537-13544 (1995); Felgner et al., 269 J Biol Chem 2550-2561(1994); Stamatatos et al., 27 Biochemistry 3917-3925 (1988); and Leventis and Silvius, 1023 Biochim Biophys Acta 124-132 (1990), and Jouani et al., 9 J. Liposome Research 95-114 (1999), which are all incorporated by reference herein.

Examples of suitable nonionic multiple lipophilic side chain amphiphilic bilayer-forming substances include, but are not limited to, glyceryl diesters, and alkoxylated amides. Examples of suitable glyceryl diesters include, but are not limited to, those glyceryl diesters having from about 10 carbon atoms to about 30 carbon atoms (e.g., from about 12 carbon atoms to about 20 carbon atoms), glyceryl dilaurate (“GDL”), glyceryl dioleate, glyceryl dimyristate, glyceryl distearate (“GDS”), glyceryl sesuioleate, glyceryl stearate lactate, and mixtures thereof, with glyceryl dilaurate, glyceryl distearate and glyceryl dimyristate, and derivatives thereof.

Examples of anionic multiple lipophilic side chain amphiphilic bilayer-forming substances include, but are not limited to, phosphatidic acids such as 1,2 dimyristoyl-sn-glycero-3-phosphate, sodium salt (DMPA), 1,2 dipalmitoyl-sn-glycero-3-phosphate, sodium salt (DPPA), 1,2 distearoyl-sn-glycero-3-phosphate, sodium salt (DSPA) and negatively charged phospholipids such as dipalmitoyl phosphatidylglycerol.

The amount of multiple lipophilic side chain amphiphilic bilayer-forming substances in the vesicle bilayer may range from, based upon the total weight of the substances in the lipid bilayer(s), from about 0.001 percent to about 95 percent (e.g., from about 5 percent to about 65 percent). The amount of multiple lipophilic side chain amphiphilic bilayer-forming substances based upon the total weight of the components in the liposome will depend upon the type of liposome (e.g., unilamellar or paucilamellar liposomes), and may range from about 0.001 percent to about 95 percent (e.g., from about 1 to about 65 percent).

A single lipophilic chain amphiphilic bilayer-forming substance is a amphililic bilayer forming substance containing a single lipophilic side chain (e.g., attached to a polar head group). The single chain lipids may be nonionic, cationic, anionic, or zwitterionic.

Examples of suitable nonionic single lipophilic chain amphiphilic bilayer-forming substances include, but are not limited to, glyceryl monoesters; polyoxyethylene fatty ethers wherein the polyoxyethylene head group has from about 2 to about 100 groups and the fatty acid tail group has from about 10 to about 26 carbon atoms; alkoxylated alcohols wherein the alkoxy group has from about 1 carbon atoms to about 200 carbon atoms and the fatty alkyl group has from about 8 carbon atom to about 30 carbon atoms (e.g., from about 10 carbon atoms to about 24 carbon atoms); alkoxylated alkyl phenols wherein the alkoxy group has from about 1 carbon atoms to about 200 carbon atoms and the fatty alkyl group has from about 8 carbon atom to about 30 carbon atoms (e.g., from about 10 carbon atoms to about 24 carbon atoms); polyoxyethylene derivatives of polyol esters; alkoxylated acids wherein the alkoxy group has from about 1 carbon atoms to about 200 carbon atoms and the fatty alkyl group has from about 8 carbon atom to about 30 carbon atoms (e.g., from about 10 carbon atoms to about 24 carbon atoms); and alkoxylated acids.

Examples of suitable glyceryl monoester nonionic single lipophilic chain amphiphilic bilayer-forming substances include, but are not limited to, those glyceryl monoesters having from about 10 carbon atoms to about 30 carbon atoms (e.g., from about 12 carbon atoms to about 20 carbon atoms), glyceryl caprate, glyceryl caprylate, glyceryl cocoate, glyceryl erucate, glyceryl hydroxystearate, glyceryl isostearate, glyceryl lanolate, glyceryl laurate, glyceryl linolate, glyceryl myristate, glyceryl oleate, glyceryl PABA, glyceryl palmitate, glyceryl ricinoleate, glyceryl stearate, and glyceryl thiglycolate, and derivatives thereof.

Examples of suitable polyoxyethylene fatty ether nonionic single lipophilic chain amphiphilic bilayer-forming substance include, but are not limited to, polyoxyethylene cetyl ether, polyoxyethylene stearyl ether, polyoxyethylene cholesterol ether, polyoxyethylene laurate, polyoxyethylene dilaurate, polyoxyethylene stearate, polyoxyethylene distearate, polyoxyethylene lauryl ether, polyoxyethylene stearyl ether, polyoxyethylene myristyl ether, and polyoxyethylene lauryl ether, e.g., with each ether having from about 3 to about 10 oxyethylene units, and derivatives thereof.

Suitable examples of an alkoxylated alcohol nonionic single lipophilic chain amphiphilic bilayer-forming substance include, but are not limited to, those having the structure shown in formula I below:

R₅—(OCH₂CH2)_{y}—OH

### Formula I

wherein R₅ is an unbranched alkyl group having from about 10 to about 24 carbon atoms and y is an integer between about 4 and about 100 (e.g., from about 10 and about 100). An example of such an alkoxylated alcohol is the species wherein R₅ is a lauryl group and y has an average value of 23, which is known by the CTFA name “laureth 23“and is available from Uniqema, Inc. of Wilmington, Del. under the tradename BRIJ 35®.

Suitable examples of an alkoxylated alkyl phenols nonionic single lipophilic chain amphiphilic bilayer-forming substance include, but are not limited to, those having the structure shown in formula II below:

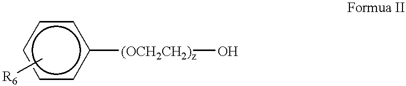

wherein R₆ is an unbranched alkyl group having from about 10 to about 24 carbon atoms and z is an integer of from about 7 and 120 (e.g., from about 10 to about 100). An example of this class of materials is the species wherein R₆ is a nonyl group and z has an average value of about 14. This material is known by the CTFA name “nonoxynol-14” and is available under the tradename, MAKON 14® from the Stepan Company of Northfield, Ill.

Suitable polyoxyethylene derivatives of polyol ester nonionic single lipophilic chain amphiphilic bilayer-forming substance are those wherein the polyoxyethylene derivative of polyol ester that: (1) is derived from (a) a fatty acid containing from about 8 to about 22 (e.g., from about 10 to about 14 carbon atoms) and (b) a polyol selected from sorbitol, sorbitan, glucose, α-methyl glucoside, polyglucose having an average of about 1 to about 3 glucose residues per molecule, glycerine, and pentaerythritol; (2) contains an average of from about 10 to about 120 (e.g., from about 20 to about 80 oxyethylene units; and (3) has an average of from about 1 to about 3 fatty acid residues per mole of polyoxyethylene derivative of polyol ester.

Examples of polyoxyethylene derivatives of polyol esters include, but are not limited to, PEG-80 sorbitan laurate and Polysorbate 20. PEG-80 sorbitan laurate, which is a sorbitan monoester of lauric acid ethoxylated with an average of about 80 moles of ethylene oxide, is available commercially from ICI Surfactants of Wilmington, Del. under the tradename Atlas G-4280®. Polysorbate 20, which is the laurate monoester of a mixture of sorbitol and sorbitol anhydrides condensed with approximately 20 moles of ethylene oxide, is available commercially from ICI Surfactants of Wilmington, Del. under the tradename Tween 20®. Another exemplary polyol ester is sorbitan stearate, which is available from Uniqema, Inc. under the tradename SPAN 60®.

Suitable examples of alkoxylated acid nonionic single lipophilic chain amphiphilic bilayer-forming substance include, but are not limited to, the esters of an acid (e.g., a fatty acid) with a polyalkylene glycol. An exemplary material of this class has the CTFA name PEG-8 laurate®.

Examples of suitable cationic single lipophilic chain amphiphilic bilayer-forming substance include, but are not limited to, quaternary trimethylmonoacyl amines wherein the acyl groups have from about 8 carbon atoms to about 30 carbon atoms (e.g., from about 10 carbon atoms to about 24 carbon atoms), and derivatives thereof such as ammonium derivatives, e.g., stearamidopropyl dimethyl (myristyl acetate) ammonium chloride (Quaternium 70), triethyl hydrogenated tallow ammonium chloride (Quaternium 16), and benzalkonium chloride, and derivatives thereof.

Examples of suitable anionic single lipophilic chain amphiphilic bilayer-forming substances include, but are not limited to, fatty acids such as oleic acid and negatively charged single chained phospholipids such as phosphatidylserine and phosphatidylglycerol.

The amount of single lipophilic chain amphiphilic bilayer-forming substance in the vesicle bilayer may range from, based upon the total weight of the substances in the lipid bilayer(s), from about 0.001 percent to about 70 percent (e.g., from about 1 percent to about 30 percent). The amount of single lipophilic chain amphiphilic bilayer-forming substance based upon the total weight of the components in the liposome will depend upon the type of liposome (e.g., unilamellar or paucilamellar liposomes), and may range from about 1 percent to about 95 percent (e.g., from about 1 percent to about 30 percent).

The above single and multiple lipophilic chain amphiphilic bilayer-forming substance may also be a phospholipid, which may be zwitterionic in nature. Examples of phospholipids include, but are not limited to, natural and synthetic phospholipids. Examples of natural phospholipids include, but are not limited to, egg phosphatidylcholine, hydrogenated egg phosphatidylcholine, soybean derived phospholipids such as soybean phosphatidylcholine, phospholipids from plant sources, sphingomyelin. Examples of synthetic phospholipids include, but are not limited to, synthetic phosphatidylcholines such as 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1,2-distearoyl-sn-glycero-3-phoshpocholine(DSPC), 1,2-didioleoyl-sn-glycero-3-phoshpocholine(DOPC), 1-palmitoyl-2-oleoyl-sn-glycero-3-phoshpocholine(POPC), phosphatidylethanolamines include, but are not limited to, 1,2-dimyristoyl-sn-glycero-3-phoshpethanolamine(DMPE), 1,2-dipalmitoyl-sn-glycero-3-phoshpethanolamine(DPPE), 1,2-distearoyl-sn-glycero-3-phoshpethanolamine(DSPE), 1,2-dioleoyl-sn-glycero-3-phoshpethanolamine(DOPE), negatively charges phospholipids such as dipalmitoyl phosphatidylglycerol (DPPG), dipalmitoyl phosphatidylcholine (DPPC), dipalmitoyl phosphatidic acid (DPPA), and phosphatidylserine (PS), and derivatives thereof.

The above single and multiple lipophilic chain amphiphilic bilayer-forming substance may also be a cermide. Examples include, but are not limited to, N-acetyl D-erythro-sphinogsine(C2Cer), N-octanoyl D-erythro-sphinogsine (C8Cer), N-myristoyl D-erythro-sphinogsine(C14Cer), N-stearoyl D-erythro-sphinogsine(C18Cer), N-arachidoyl D-erythrosphinogsine(C20Cer).

Other suitable lipids are further described in the following references: Avanti Polar Lipids, Inc., Alabaster, AL Interim Catalog 13-92 and 105-127 (1999); polyglycerol such as those described in U.S. Pat. No. 4,772,471, French Patent Nos. 1,477,048 and 2,091,516;; amide-based oligomeric cationic lipids such as those described in U.S. Pat. No. 5,877,220; cationic lipids such as those described in U.S. Pat. Nos. 5,980,935, 5,851,548, 5,830,430, and 5,777,153; phosphonic acid-based cationic lipids such as those described in U.S. Pat. No. 5,958,901; quaternary cytofectins such as those described in U.S. Pat. No. 5,994,317; ether lipids such as those described in U.S. Pat. No. 5,989,587; and polyethylene glycol modified cermide lipids such as those described in U.S. Pat. No. 5,820,873.

Sterols may be added to the lipid bilayer of the liposome. The presence of a rigid steroid alongside the fatty acid chains of the lipid in the bilayer may reduce the freedom of motion of these carbon chains, creating a better packing of the lipid bilayers. Examples of suitable sterols include, but are not limited to, cholesterol and salts and esters thereof, cholesterol 3-sulfate, phytocholesterol, hydrocortisone, alpha-tocopherol, betasitosterol, bisabolol and derivatives thereof.

The amount of sterol in the vesicle bilayer may range from, based upon the total weight of the substances in the vesicle bilayer, from about 0.001 percent to about 95 percent (e.g., from about 1 percent to about 65 percent). The amount of sterol, based upon the total weight of the components in the liposome will depend upon the type of liposome (e.g., unilamellar or paucilamellar liposomes), and may range from about 0.001 percent to about 95 percent (e.g., from about 1 percent to about 65 percent).

The liposomes manufactured by the present method may contain a benefit agent (e.g., a cosmetic, diagnostic, or pharmaceutical agent). Examples of benefit agents include, but are not limited to, those suitable for treating the symptoms and/or the disorders of the skin and hair (e.g., hair loss or growth, dandruff, seborrheic dermatitis and/or psoriasis, fine lines and wrinkles, pigmentation).

Examples of benefit agents include, but are not limited to, azoles such as elubiol and ketoconazole; shale oil and derivatives thereof; coal tar; salicylic acid; zinc pyrithione; selenium sulfide; hydrocortisone; sulfur; menthol; pramoxine hydrochloride; potassium channel openers or peripheral vasodilators such as minoxidil, diazoxide, and compounds such as N*-cyano-N-(tert-pentyl)-N′-3-pyridinyl-guanidine (“P-1075”); vitamins such as vitamin A, vitamins B, vitamin E, vitamin K, and vitamin C, and derivatives thereof (e.g., retinoids such as retinol, retinoic acid, isotretinoin, retinal, retinyl palmitate, and retinyl acetate, vitamin E acetate and vitamin C palmitate); hormones such as erythropoietin, prostaglandins (e.g., prostaglandin El and prostaglandin F2-alpha); fatty acids such as oleic acid; diruretics such as spironolactone; heat shock proteins (‘HSP”) such as HSP 27 and HSP 72; calcium channel blockers such as verapamil HCL, nifedipine, and diltiazemamiloride; immunosuppressant drugs such as cyclosporin and Fk-506; 5 alpha-reductase inhibitors such as finasteride; growth factors such as EGF, IGF and FGF; transforming growth factor beta; tumor necrosis factor; non-steroidal anti-inflammatory agents such as benoxaprofen; cell adhesion molecules such as ICAM; glucorcorticoids such as betametasone; botanical extracts such as aloe, clove, ginseng, rehmannia, swertia, sweet orange, zanthoxylum, Serenoa repens (saw palmetto), Hypoxis rooperi, stinging nettle, pumpkin seeds, rye pollen, sandlewood, red beet root, chrysanthemum, rosemary, and burdock root; homeopathic agents such as Kalium Phosphoricum D2, Azadirachta indica D2, and Joborandi DI; genes; antibiotics such as streptomycin; proteins inhibitors such as cycloheximide; acetazolamide; benoxaprofen; cortisone; diltiazem; hexachlorobenzene; hydantoin; nifedipine; penicillamine; phenothaiazines; pinacidil; psoralens, verapamil; zidovudine; alpha-glucosylated rutins; antineoplastic agents such as doxorubicin, cyclophosphamide, chlormethine, methotrexate, fluorouracil, vincristine, daunorubicin, bleomycin and hydroxycarbamide; anticoagulants such as heparin, heparinoids, coumaerins, detran and indandiones; antithyroid drugs such as iodine, thiouracils and carbimazole; lithium and lithium carbonate; interferons, such as interferon alpha, interferon alpha-2a and interferon alpha-2b; glucocorticoids such as betamethasone, and dexamethosone; antihyperlipidaemic drugs such as triparanol and clofibrate; thallium; mercury; albendazole; allopurinol; amiodarone; amphetamines; androgens; bromocriptine; butyrophenones; carbamazepine; cholestyramine; cimetidine; clofibrate; danazol; desipramine; dixyrazine; ethambutol; etionamide; fluoxetine; gentamicin, gold salts; hydantoins; ibuprofen; impramine; immunoglobulins; indandiones; indomethacin; intraconazole; levadopa; maprotiline; methysergide; metoprolol; metyrapone; nadolol; nicotinic acid; potassium thiocyanate; propranolol; pyridostimine; salicylates; sulfasalazine; terfenadine; thiamphenicol; thiouracils; trimethadione; troparanol; valproic acid; inorganic sunscreens such as titanium dioxide and zinc oxide; organic sunscreens such as octyl-methyl cinnamates and derivatives thereof; antioxidants including beta carotene, alpha hydroxy acid such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucopehtonic acid, glucopheptono 1,4-lactone, gluconic acid, gluconolactone, glucuronic acid, glucurronolactone, glycolic acid, isopropyl pyruvate, methyl pyruvate, mucic acid, pyruvia acid, saccharic acid, saccaric acid 1,4-lactone, tartaric acid, and tartronic acid; beta hydroxy acids such as beta-hydroxybutyric acid, beta-phenyl-lactic acid, and beta-phenylpyruvic acid; resorcinol; antibiotics such as tetracycline, erythromycin, and the anti-inflammatory agents such as ibuprofen, naproxen, hetprofen; kojic acid and its derivatives such as, for example, kojic dipalmitate; hydroquinone and it derivatives such as arbutin; transexamic acid; azelaic acid; placertia; and licorice; and derivatives thereof.

The benefit agent may be contained within the lipid bilayer (e.g., if it is a lipophilic agent) or within a hydrophilic component of the liposome (e.g., within the hydrophilic regions within the lipid bilayers of within the core). The hydrophilic component may contain water and/or other polar solvents. Examples of polar solvents include, but are not limited to, glycols such as glycerin, alcohols (e.g., those alcohols having from about 2 carbon atoms to about 6 carbon atoms), propylene glycol, sorbitol, oxyalkylene polymers such as PEG 4, and derivatives thereof.

The liposomes of the present invention may be included within pharmaceutical (e.g., compounded with a pharmaceutically compatible carrier) or a cosmetic (e.g., compounded with a cosmetically acceptable carrier). The resulting composition may be in the form of a cream, ointment, lotion, gel, or shampoo for therapeutic or cosmetic use.

The following is a description of the manufacture and testing of liposomes of the present invention. Other liposomes of the invention can be prepared in an analogous manner by a person of ordinary skill in the art.

### EXAMPLE 1

### Preparation of Liposomes

Table 1 describes the ingredients (based upon weight percentages of the entire phase) of the six multilamellar liposome formulations used in the subsequent examples.

TABLE 1

| Formulations # | 1 | 2 | 3 | 4 | 5 | 6 |
|---|---|---|---|---|---|---|
| Lipid Phase | | | | | | |
| Glyceryl | 12.3 | 10.8 | 3.30 | 10.8 | 10.8 | 0 |
| distearate | | | | | | |
| Glyceryl dilaurate | 0 | 0 | 7.50 | 0 | 0 | 10.8 |
| Cholesterol | | | | | | |
| Polyoxyethylene- | 4.1 | 3.6 | 3.6 | 3.6 | 3.6 | 3.60 |
| 10-stearyl ether | | | | | | |
| Cationic lipid* | 10.9 | 9.6 | 9.6 | 9.6 | 9.6 | 9.60 |
| Dichiorophenyl | | | | | | |
| Imadazol- | 0 | 6.0 | 6.00 | 6.00 | 6.00 | 6.00 |
| dioxolan | 2.7 | 0 | 0 | 0 | 0 | 0 |
| Aqueous Phase | | | | | | |
| Zinc Pyrithione | 0 | 15.6 | 15.60 | 20.83 | 31.25 | 15.6 |
| (48%) | | | | | | |
| Zinc | 6.0 | 0 | 0 | 0 | 0 | 0 |
| Pyrithione | | | | | | |
| (100%) | | | | | | |
| Methyl Paraben | 0 | 0.20 | 0.20 | 0.20 | 0.20 | 0.20 |
| Propyl Paraben | 0 | 0.05 | 0.05 | 0.05 | 0.05 | 0.05 |
| Sodium Citrate | 0 | 0.15 | 0 | 0.15 | 0.15 | 0 |
| Dionized water | 64.0 | 54.0 | 54.15 | 48.78 | 54.80 | 54.15 |
| Total | 100 | 100 | 100 | 100 | 100 | 100.0 |

*Cationic lipid used was di (soyoylethyl) hydroxyethylmonium methosulfate (DSHM).

Each of the above formulations were made by the following four manufacturing methods: the Sonolator” Method of the present invention and the three previous well-known methods of making liposomes using the Gaulin Homongenizer, MVS System, or syringes.

1. Sonolator™ Method: The appropriate amounts of the lipid phase ingredients were mixed in a beaker at 65° C. until the lipids melted. The aqueous phase ingredients were then mixed and heated to 60° C. The resulting hot liquid phases were then each poured into separate aqueous and oil phase reservoirs of the Sonolator™ machine Model No. A-HP. The feed line valves for each feed line were then opened and the feed pumps were started. The operating pressure, orifice size, and cooling rates were established as set forth in the examples below. The flow rate was estiblished at 3 parts lipid phase to 7 parts aqueous phase. The attenuation was adjusted with the tuning valve and the distance of the blade from the orifice were adjusted to record the maximum oscillation reading (i.e., 25,000 MHz).

2. The Gaulin Method:

The appropriate amounts of the lipid phase ingredients were mixed in a beaker at 65° C. until the lipids melted. The aqueous phase ingredients were mixed and heated to 600C. The resulting hot phases were then each poured into separate aqueous or oil phase reservoirs. The oil phase and the aqueous phase were delivered into the Gaulin Homogenizer, 15 15MP-8TBS, APV Gaulin, Everett, Mass., by gravity feed. The lipid reservoir was located above the aqueous compartment in order to eliminate back flow. The two phases met at a single opening before entering the mixing compartment. After a feed rate of 3 parts lipid phase to 7 parts aqueous phase was established, the feed lines were open under a pressure set by the operator to a pressure of either 1800 or 4200 psi. The two phases flowed into the mixing chamber under pressure through a restricted opening that created the shear forces to produce the liposomes. The product was then collect.

3. The MVS System Method:

The appropriate amounts of the lipid phase ingredients were mixed in a beaker at 65° C. until the lipids melted. The aqueous phase ingredients were then mixed and heated to 60° C. The resulting hot phases were then poured into separate aqueous or oil phase reservoirs of the MVS machine, IGI Inc., Buena, N.J. The positive displacement pump for the lipid and aqueous feed lines were then turned on. After the feed rate of 3 parts lipid phase to 7 part aqueous phase was established, the valves to the feed lines were opened and the aqueous phase and lipid phase were transported from injection jets into a cylindrical mixing chamber. The resulting liposomes were then withdrawn through an exit tube.

4. The Svringe Method: The appropriate amounts of the lipid phase ingredients were mixed in a beaker at 75° C. until the lipids melted. The resulting melt was then drawn into a syringe, which was preheated in a water-bath to 75° C. A second syringe containing appropriate amounts of the hydrophilic component was preheated in a water-bath to 70° C. The two syringes were then connected via a 3-way metal stopcock. The ratio of aqueous phase to lipid phase was about 70:30 or 7 ml of aqueous phase to 3 ml of lipid phase. After injecting the hydrophilic component into the lipid phase syringe, the resulting mixture was rapidly mixed back and forth between the two syringes several times until the contents cooled to about 25-30° C.

### Example 2

### Freeze Fracture Microscopy

The six compositions of Examples 1 were each prepared by the above four methods and were subsequently examined using a freeze-fracture transmission electron microscope (FF-TEM). FF-TEM samples of each formulation were prepared in accordance with techniques described in chapter 5 of “Low Temperature Microscopy and Analysis” by Patrick Echlin (1992). The samples were fractured at low temperature and etched at −150° C. for purposes of removing a surface layer of water.

Liposomes of Example 1 manufactured by the Syringe Method showed the presence of large bilayered structures ranging in size from 100 nm to 400 nm. Upon accelerated aging at 50° C. for 4 weeks, these vesicles slightly increased in size.

Liposomes of Example 1 manufactured by the MVS System method showed the presence of intact vesicles with bilayers. However, upon accelerated aging at 3 weeks at 50° C., the vesicles doubled in size.

Liposomes of Example 1 manufactured using the Sonolator™ method were very intact both at the initial time point and upon accelerated aging at 50° C. for 4 weeks.

Liposomes of Example 1 manufactured using the Gaulin method were intact at the initial time point, but they were not checked for further stability.

### Example 3

### Determination of Entrapment of Agents

The degree of zinc pyrithione entrapment in the liposomes was determined using size exclusion chromatography with Sephadex G-75 columns, Sigma Chemical Co., St. Luis, Mo. Details of this procedure is set forth in Dowton, S. M., et al, 1993 “Influence of liposomal composition on topical delivery of encapsulated cyclosporin A I. An in vitro study using hairless mouse skin,” STP Pharma Sci., 3, 404-407. The liposomal formulations from Example 1 were tested for zinc pyrithione entrapment under accelerated stability conditions. Tables 4 through 8 below shows the level of entrapment of the benefit agent ZPT for each formulation tested.

The effect of pressure on the entrapment of ZPT in the liposomes made via the Sonolator™ using Formulation 2 From Table 1 is shown in Table 4 and 5. The orifice size was set at 0.00072 in² and cooled under ambient tempertures.

TABLE 4

| Pressure (psi) | % Entrapment |
|---|---|
| 500 | 96.1 |
| 1500 | 91.4 |
| 2000 | 68.4 |

It is evident that the effect of pressure is major factor on the entrapment of the active in the vesicles. The discovered trend, however, is surprising since the increased pressure should have resulted in higher shear and a high percentage of ZPT entrapment in the vesicles. In conventional liposomal manufacturing methods, higher shear forces results in higher entrapment values of active agents. However, it was surprisingly found that the opposite occurred using the Sonilator™ Method. At the higher pressures, the amount of ZPT entrapped inside the vesicles decreased. This finding may indicate that at higher pressures, the lipids formed other structures such as micelles and liquid crystal lattices that prevented ZPT from being encapsulated in the core of the liposomes.

The effect of lipid composition in the liposomes is illustrated below in Table 6.

TABLE 6

| | Major Lipid | Orifice | Pressure | |
|---|---|---|---|---|
| Formulation | Component | size | (psi) | % Entrapment |
| 6 | GDL | 0.00105 in² | 1500 | 81.6 |
| 3 | GDS/GDL | 0.00105 in² | 1500 | 86.4 |

As indicated in Table 6 above, the effect of lipid composition is an important parameter. By reducing the chain length of the major lipid in the composition, glyceryl distearate, which has a C18 carbon chain length, to a mixture of lipids that have C18 and C12 (e.g. glyceryl disterate and glyceryl dilaurate) chain lengths, the entrapment of ZPT in the liposomes decreased only slightly.

This finding is unexpected since the decrease in chain length would normally lead to a dramatic decrease in ZPT entrapment. It is known that decreasing the chain lengths of the liposomal components below C18 leads to a decrease in entrapment since the bilayers become less stable and more fluid in nature. The optimized chain lengths of lipids in a liposomal bilayers ranges between C18 to C24. Hence, the decrease of entrapment of only 5% is very unexpected, as a much higher decrease would be expected due to the increase in fluidity of the liposomal bilayers. This result indicates that the manufacturing process provides a method of preparing more uniform and stable vesicles since the entrapment of ZPT was only slightly decreased.

Next, the effect of the amount of active loaded in the liposomes is shown in Table 7 below at two different sets of pressures (using the same orifice size of 0.00072 in² and cooling rate of 65° C. to 50° C.). Formulation 2 was modified by adjusting the ZPT concentrations accordingly.

TABLE 7

| | Pressure | |
|---|---|---|
| % ZPT | (psi) | % Entrapment |
| 7.5 | 750 | 88.65 |
| 10.0 | 600 | 84.41 |
| 15.0 | 850 | 78.61 |
| 7.5 | 1500 | 75.86 |
| 10.0 | 1200 | 98.85 |
| 15.0 | 1650 | 96.97 |

Usually the effect of loading is that there is a point of saturated of the vesicles for the entrapment of actives. This saturating point is different for each active and depends upon the physical-chemical nature of the individual active to be entrapped. The effect of the lower pressures, between 600-850 psi, appears to be constant with respect to the entrapment of ZPT. That is, more ZPT is equally entrapped at relatively similar pressures. However, at higher pressures the effect of loading becomes more apparent. This is unexpected since one would expect, that regardless of the pressure, the loading of the active would outweigh the pressure effect since saturation capacity is determine by the active loading, the active physical-chemical nature and the vesicles composition. It is evident that at certain pressures the entrapment of the active is independent. Hence, at certain pressures with a maximum loading of active one could optimize the entrapment of the active.

Lastly, the effect of the manufacturing method of preparing liposomes of Table 1 is shown below in Table 8.

TABLE 8

| Formulation | Manufacturing | Pressure | |
|---|---|---|---|
| No. | Method | (psi) | % Entrapment |
| 6.0 | Syringe Method | NA | 87.25 |
| 7.5 | MSV System | NA | 71.89 |
| Method | | | |
| 7.5 | Gaulin Method | 1800 | 72.16 |
| | | 4200 | 67.83 |
| 7.5 | Sonolator ™ | 500 | 96.13 |
| | | 1500 | 91.35 |

The Sonoloator™ Method produced the highest percentage of entrapped ZPT. The syringe method had a lower loading of active and did not achieve the high levels as the Sonolator™ Method. The MVS and Gaulin methods performed the less efficiently with respect to ZPT entrapment at similar loading.

### Example 4

### Particle Size Analysis

After preparing the compositions in accordance with Examples 1, the particle sizes of the resulting formulations of Formulation #2 were analyzed by inserting 5 1 ml of a 10-fold dilution of each formulation into a Nicomp 370-submicron particle analyzer, Nicomp Particle Sizing Systems, Santa Barbara, Calif. using dynamic laser light scattering. The results are presented in Tables 9 below, which shows the size ranges and distribution type (e.g., unimodal, bimodal, or trimodal distribution) based on number-weighted mean diameter of the vesicles of Formulation #2 of Example 1 made via four different manufacturing methods. The Nicomp 370 is unable to accurately detect particle ranges below 30-nm (limit of detection is 20 nm) and vesicles larger than 30 μm (30,000 nm).

The results of the particle size data from liposomes made via different manufacturing methods and placed upon accelerated stability are further shown in Table 9 below.

TABLE 9

| | | Manu- | Number | % in |
|---|---|---|---|---|
| Duration & | Distri- | facturing | Distribution | Population |
| Condition | bution | Method | (nm) | (Based on #) |
| Initial | Trimodal | Syringe | 347.8 ± 42.9 | 96.4 |
| | | Method | 3158.2 ± 375.9 | 3.5 |
| | | | 26327 ± 2313 | 0.1 |
| 4 weeks @ | Bimodal | Syringe | 775.4 ± 94.4 | 93.4 |
| 50° C. | | Method | 6381.8 ± 1055 | 6.6 |
| Initial | Unimodal | MVS | 241.1 ± 24.1 | 100 |
| | | Machine | | |
| 3 weeks @ | Unimodal | MVS | 896.6 ± 57.5 | 100 |
| 50° C. | Machine | | | |
| Initial | Bimodal | Sonolator ™ | 111.8 ± 9.6 | 94.7 |
| | | | 602.9 ± 84.5 | 5.3 |
| 4 weeks @ | Trimodal | Sonolator ™ | 134.3 ± 18.6 | 86.8 |
| 50° C. | | | 554.6 ± 92.0 | |
| | | | 5024 ± 461.3 | 13.0 |
| | | | | 0.2 |

As shown in the Table 9, the major particle size distribution of the vesicles ranged from 0.111 to 0.896 μm. The vesicles made via the syringe method as described in Example 1 increased in size over 2 fold during accelerated aging at 50° C. over 4 weeks. The liposomes made via the MVS System method showed a similar trend. At 3 weeks at accelerated stability at 50° C., the size of the liposomes increased 3.7 times indicating the MVS System method may lead to an instability in the size of the liposomes over time and eventually lead to phase separation of the product. The liposomes made via the Sonolator^{Tm} remained extremely stable over 4 weeks at 50° C. Over 86% of the liposomes was between 115 to 152 nm in diameter. This indicated that the liposomes are very stable and there are no apparent stability issues. These results are further supported by the freeze-fracture micrographs from Example 2.

This example illustrated that the particle size of the liposomes made via the Sonolator™ remained relatively constant over time upon accelerated storage conditions, whereas other method of manufacturing lead to significance increase in the size of the liposomes under the same conditions. Trimodal distribution indicates that there are 3 distinct populations of vesicles with different sizes. Bimodal distribution is two district populations of vesicles and unimodal indicates only one population of vesicles with relatively the same size.

It is understood that while the invention has been described in conjunction with the detailed description thereof, that the foregoing description is intended to illustrate and not limit the scope of the invention, which is defined by the scope of the appended claims.

Other aspects, advantages, and modifications are within the claims.

What is claimed is:

1. A method of making a liposome, said method comprising the steps of:
(a) mixing a lipophilic phase and a hydrophilic phase, said lipophilic phase comprising an amphiphilic bilayer-forming substance; and
(b) applying a cavitational sheer force to said mixture to form said liposome;
wherein said cavitational force is created by contacting said mixture with a member that vibrates at an ultrasonic frequency.

2. A method of claim 1, wherein said mixture is contacted with said member under pressure ranging from about 2 psi to about 10,000 psi.

3. A method of claim 1, wherein said member is in the form of a blade.

4. A method of claim 2, wherein said member is in the form of a blade.

5. A method of claim 1, wherein said method further comprises the step of applying a second sheer force to said mixture following said cavitational sheer force, wherein said second sheer force is created by passing said mixture through an orifice.

6. A method of claim 4, wherein said method further comprises the step of applying a second sheer force to said mixture following said cavitational sheer force, wherein said second sheer force is created by passing said mixture through an orifice.

7. A method of claim 1, wherein said lipophilic phase comprises both a multiple lipophilic side chain amphiphilic bilayer-forming substances and a single lipophilic side chain amphiphilic bilayer-forming substances.

8. A method of claim 1, wherein said lipophilic phase further comprises a sterol.

9. A method of claim 7, wherein said lipophilic phase further comprises a sterol.

10. A method of claim 6, wherein said lipophilic phase comprises a multiple lipophilic side chain amphiphilic bilayer-forming substances, a single lipophilic side chain amphiphilic bilayer-forming substances, and a sterol.

11. A method of claim 1, wherein said liposome is a multilamellar liposome.

12. A method of claim 6, wherein said liposome is a multilamellar liposome.

13. A method of claim 1, wherein said liposome is a paucilamellar liposome.

14. A method of claim 6, wherein said liposome is a paucilamellar liposome.

15. A method of claim 1, wherein said mixture further comprises a benefit agent.

16. A method of claim 6, wherein said mixture further comprises a benefit agent.

17. A method of claim 7, wherein said mixture further comprises a benefit agent.

18. A method of claim 8, wherein said mixture further comprises a benefit agent.

19. A method of claim 11, wherein said mixture further comprises a benefit agent.

20. A method of claim 13, wherein said mixture further comprises a benefit agent.
